(19) United States
(12) Patent Application Publication
(10) Pub. No.: US 2013/0218017 A1
(43) Pub. Date: Aug. 22, 2013

(54) PORTABLE ULTRASONIC DIAGNOSTIC APPARATUS

(71) Applicant: SAMSUNG MEDISON CO., LTD., (US)

(72) Inventors: Soon Deok Kim, Gyeonggi-do (KR); Jung Sik Song, Seoul (KR)

(73) Assignee: SAMSUNG MEDISON CO., LTD., Gangwon-Do (KR)

(21) Appl. No.: 13/769,065

(22) Filed: Feb. 15, 2013

(30) Foreign Application Priority Data
Feb. 17, 2012 (KR) 10-2012-0016552

(51) Int. Cl. A61B 8/00

(52) U.S. Cl. CPC A61B 8/4427; A61B 8/4444; A61B 8/54; A61B 8/462; A61B 8/467
USPC 600/459; 600/437

(57) ABSTRACT

A portable ultrasonic diagnostic apparatus includes a body, a handle installed at a side of the body and configured for protruding from or retracting into the body, and a display unit displaying an image. The display unit is detachably mounted on the handle, and thus the handle installed to the body functions as a stand for the display unit. Thus, it is convenient to use the portable ultrasonic diagnostic apparatus.

### CROSS-REFERENCE TO RELATED APPLICATION(S)

This application claims the benefit of priority to Korean Patent Application No. 2012-0016552, filed on Feb. 17, 2012 in the Korean Intellectual Property Office, the disclosure of which is incorporated herein by reference.

### 1. TECHNICAL FIELD

Present application relates to a portable ultrasonic diagnostic apparatus that is easily carried by a user.

### 2. BACKGROUND

Generally, an ultrasonic diagnostic apparatus is an apparatus that emits ultrasonic waves from the surface of the body of an object to an internal body region to be diagnosed and acquires tomograms of soft tissue and images of blood flow via the ultrasonic waves reflected from the body of the object.

A portable ultrasonic diagnostic apparatus includes a display unit including a display provided at the front surface thereof, a probe connected to the display unit and transmitting and receiving ultrasonic waves, a body accommodating the display unit and a control unit to control operation of the apparatus.

The display unit needs to be placed on a stand, in order to use such a portable ultrasonic diagnostic apparatus more conveniently. However, it is not easy to carry the stand due to large volume thereof. A need exists for a way to easily hold a display unit to the portable diagnostic apparatus.

### SUMMARY

An aspect of the present disclosure encompasses a portable ultrasonic diagnostic apparatus that is convenient to use.

Additional aspects of the disclosure will be set forth in part in the description which follows and, in part, will be obvious from the description, or may be learned by practice of the invention.

One aspect of the present disclosure relates to a portable ultrasonic diagnostic apparatus including a body, a handle installed at a side of the body and configured for protruding from or retracting into the body, and a display unit detachably mounted on the handle. The portable ultrasonic diagnostic apparatus further includes wheels protruding downward from the body and supported by the ground

The portable ultrasonic diagnostic apparatus may further include a probe connected to the body or the display unit, transmitting ultrasonic waves to an object, and receiving ultrasonic waves from the object.

The body may include a case defining an appearance of the body and the display unit may be accommodated by the case.

The body may include a control unit provided in the case.

The handle may include a handle portion for gripping and at least one elevation portion that is vertically collapsible to the body and connected to the handle portion via an upper end, and the display unit may be detachably mounted on the elevation portion.

The at least one elevation portion may include a pair of elevation portions extending in parallel to each other and connected to the handle portion via upper ends.

The portable ultrasonic diagnostic apparatus may further include a hinge assembly disposed between the display unit and the elevation portion to allow the display unit to be rotatably coupled to the handle.

The hinge assembly may include a first hinge bracket mounted on the rear surface of the display unit, a second hinge bracket coupled to the handle, and a hinge member coupled to the first hinge bracket so as to be rotatable upward or downward and coupled to the second hinge bracket so as to be rotatable clockwise or counterclockwise.

The second hinge bracket may include an engagement protrusion protruding backward and coupled to the elevation portion, and the elevation portion may have an engagement hole to which the engagement protrusion is coupled.

The case may include a probe holder that rotatably protrudes from the case and holds the probe.

The probe holder may have one end rotatably attached to a side of the case such that the probe holder is rotatably collapsed to form a surface of the case, and protrudes rotatably from the case as the probe holder rotates.

The display unit may include a display disposed at the front surface of the display unit and displaying an image.

The display may include a touch-screen.

The body may further include an auxiliary handle disposed at one side of the body.

The case may have a recessed platform on an upper surface.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a perspective view illustrating a portable ultrasonic diagnostic apparatus according to an embodiment of the present disclosure;

FIG. 2 is a perspective view illustrating a portable ultrasonic diagnostic apparatus according to an embodiment of the present disclosure in use;

FIG. 3 is a perspective view illustrating a display unit of a portable ultrasonic diagnostic apparatus according to an embodiment of the present disclosure mounted on a handle; and

FIG. 4 is a perspective view illustrating a display unit of a portable ultrasonic diagnostic apparatus according to another embodiment of the present disclosure mounted on a handle.

### DETAILED DESCRIPTION

Reference will now be made in detail to the embodiments of the present disclosure, examples of which are illustrated in the accompanying drawings, wherein like reference numerals refer to like elements throughout.

Hereinafter, a portable ultrasonic diagnostic apparatus according to an embodiment of the present disclosure will be described in detail.

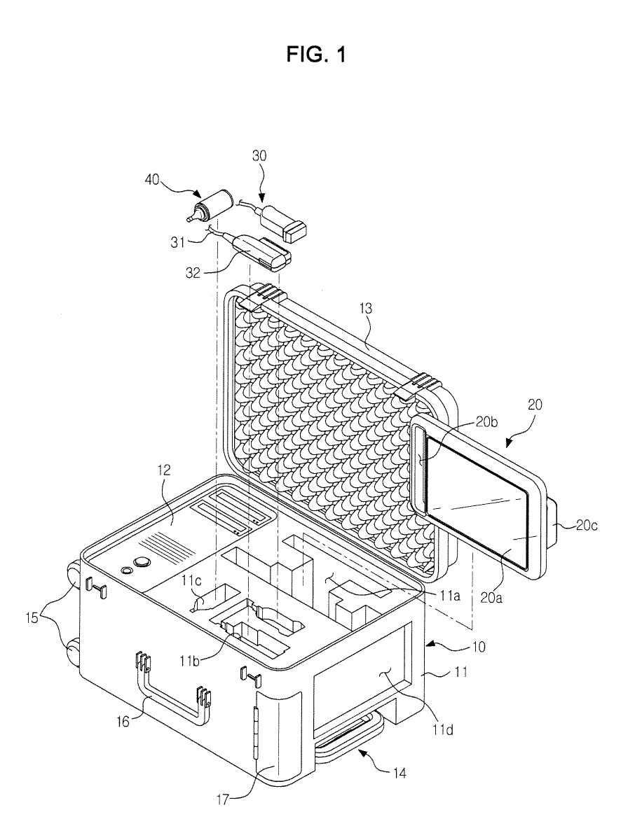

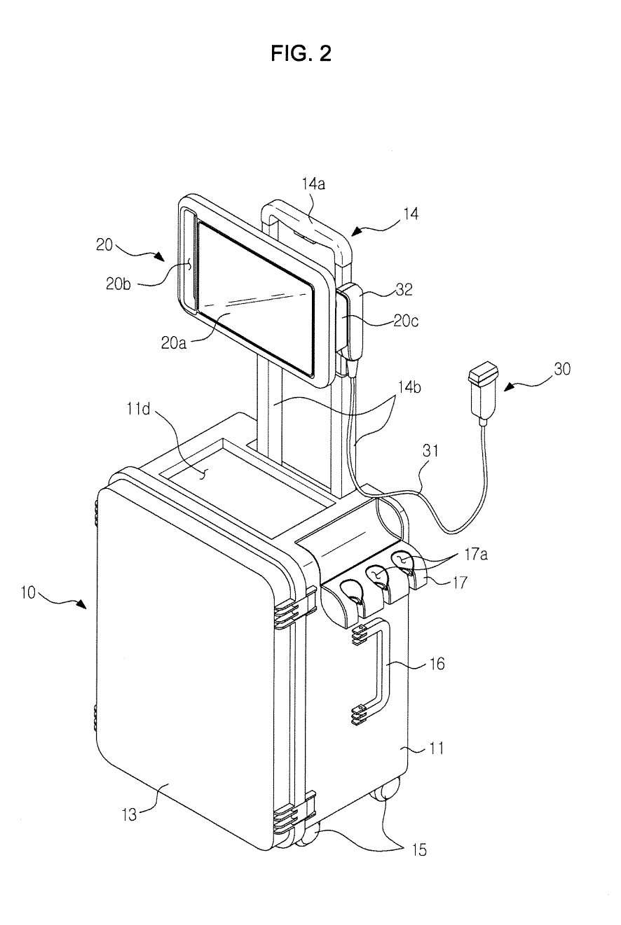

Referring to FIGS. 1 and 2, the portable ultrasonic diagnostic apparatus includes a body 10, a display unit 20, and a probe 30. The display unit 20 includes a display 20a disposed at the front surface thereof and displaying an image such as diagnosis results. The probe 30 is connected to the display unit 20, transmits ultrasonic waves to an object to be diagnosed, and receives ultrasonic waves reflected by the object.

Referring to FIGS. 1 and 2, the body 10 includes a case 11 defining an appearance thereof, and a control unit 12 disposed in the case 11 and controlling operation of the portable ultrasonic diagnostic apparatus. The display unit 20 and the probe 30 may be accommodated in the case 11.

The display 20a is provided at the front surface of the display unit 20 as described above. According to the present embodiment, the display 20a is a touch-screen such that a user may control operation of the portable ultrasonic diagnostic apparatus via the touch of the display 20a. In addition, the display unit 20 has a handle groove 20b at a side thereof to allow the user to easily hold the display unit 20 and includes a terminal 20c disposed at the rear surface of the display unit 20 and connected to the probe 30.

The probe 30 is detachably connected to the display unit 20. To this end, the probe 30 includes a cable 31 having a predetermined length and a connector 32 disposed at one end of the cable 31 to connect to the terminal 20c.

The case 11, one side of which is open, has a plurality of accommodation portions 11a, 11b, and 11c to accommodate the display unit 20, the probe 30, and the like. A cover 13 has one end rotatably coupled to one side of the case 11, and opens and closes the accommodation portions 11a, 11b, and 11c as the cover 13 rotates with respect to the case 11. The case 11 includes a handle 14 that is movably disposed on one side of the case 11, and retracts downward into and protrudes upward from the case 11 to allow the user to pull the body 10, a plurality of wheels 15 that protrude downward from the case 11 and are supported by the ground, and an auxiliary handle 16 that is disposed at one side of the case 11 to allow the user to carry the body 10.

The case 11 may further accommodate a gel container 40 containing ultrasound gel used in ultrasonic diagnosis and any other accessories in addition to the display unit 20 and the probe 30. The accommodation portions 11a, 11b, and 11c may include a first accommodation portion 11a to accommodate the display unit 20, a second accommodation portion 11b to accommodate the probe 30, a third accommodation portion 11c to accommodate the gel container 40, and the like.

The case 11 includes a recessed platform 11d on which accessories used for ultrasonic diagnosis such as the gel container 40 are placed, and a probe holder 17 protruding from one side of the case 11 and housing the probe 30. The probe holder 17 has one end rotatably attached to the case 11, and restores into or projects away from the case 11 as the probe holder 17 rotates with respect to the case 11. The probe holder 17 has a plurality of probe holding grooves 17a in which the probe 30 is held.

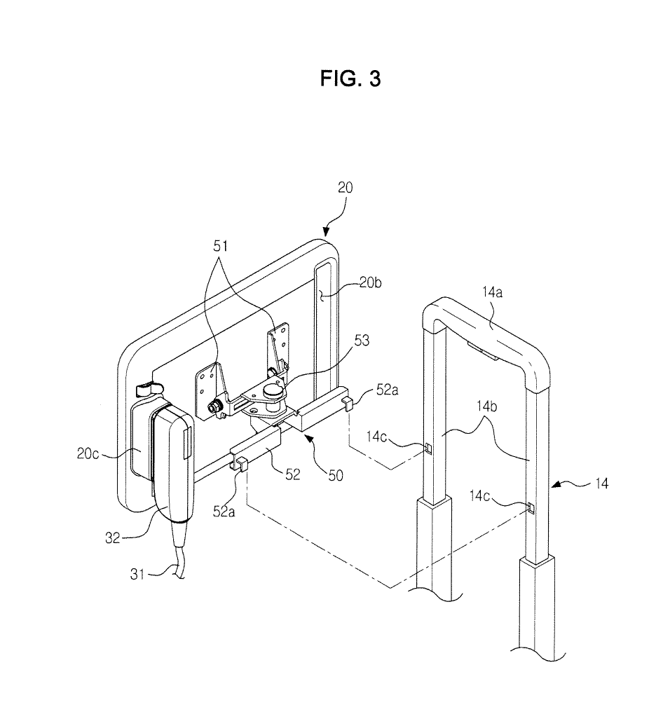

Referring to FIG. 3, the handle 14 installed to the side of the case 11 is vertically movable to allow the user to selectively adjust the length of a protruding portion of the handle 14. The handle 14 includes a handle portion 14a to allow the user to grip the handle 14 and pull the case 11 and an elevation portion 14b vertically movable along the side of the case 11 and protruding upward from the case 11. The handle portion 14a is connected to an upper end of the elevation portion 14b. According to the present example, the elevation portion 14b includes a pair of elevation portions 14b disposed in parallel to each other, and upper ends of both elevation portions 14b are connected to both ends of the handle portion 14a.

The display unit 20 is detachably mounted on the handle 14 provided to the body 10, and thus the body 10 may be used as a stand to support the display unit 20. That is, since the display unit 20 can be attached to the elevation portions 14b of the handle 14 protruding upward from the case 11, the display unit 20 may be kept at a predetermined height. In this regard, the height of the display unit 20 may be adjusted by moving the elevation portions 14b of the handle 14 to which the display unit 20 is mounted upward or downward since the handle 14 is vertically movable along the one side of the case 11.

Since the display unit 20 is rotatably installed with respect to the handle 14, the display unit 20 may be rotated within a predetermined range. To this end, a hinge assembly 50 is disposed between the display unit 20 and the handle 14 as shown in FIG. 3, so that the display unit 20 is rotatably installed to the handle 14 by the hinge assembly 50.

The hinge assembly 50 includes a pair of first hinge brackets 51 installed at opposite sides of the rear surface of the display unit 20, a pair of second hinge brackets 52 detachably attached to the elevation portions 14b, and a hinge member 53 having both ends coupled to the first hinge brackets 51 so as to be rotatable upward or downward and a central portion coupled to the second hinge brackets so as to be rotatable clockwise or counterclockwise. Accordingly, the display unit 20 may be rotatably attached to the elevation portions 14b of the handle 14 via the hinge assembly 50 so as to be rotatable upward or downward or clockwise or counterclockwise.

In order to detachably install the hinge assembly 50 to the elevation portions 14b, each of the second hinge brackets 52 has an engagement protrusion 52a, which protrudes backward, and is coupled to each of the elevation portions 14b, and each of the elevation portions 14b has an engagement hole 14c to which the engagement protrusion 52a is coupled. Thus, the hinge assembly 50 and the display unit 20 are mounted on the handle 14 by coupling the engagement protrusion 52a of the hinge assembly 50 to the engagement hole 14c of the elevation portion 14b. The hinge assembly 50 and the display unit 20 are separated from the handle 14 by detaching the engagement protrusion 52a from the engagement hole 14c.

According to the present embodiment, the display unit 20 is fixed to the hinge assembly 50, and the hinge assembly 50 is coupled to the handle 14 via the engagement protrusion 52a formed at the rear surface of the display unit 20. However, the disclosure is not limited thereto, and the display unit 20 may also be directly coupled to the handle 14 by forming the engagement protrusion 52a at the display unit 20.

In addition, the display 20a of the display unit 20 applied to the portable ultrasonic diagnostic apparatus is a touch-screen, and the display unit 20 is controlled via the display 20a according to the present embodiment. However, the disclosure is not limited thereto, and the display unit 20 may also be controlled by a control panel (not shown) separately formed from the display unit 20.

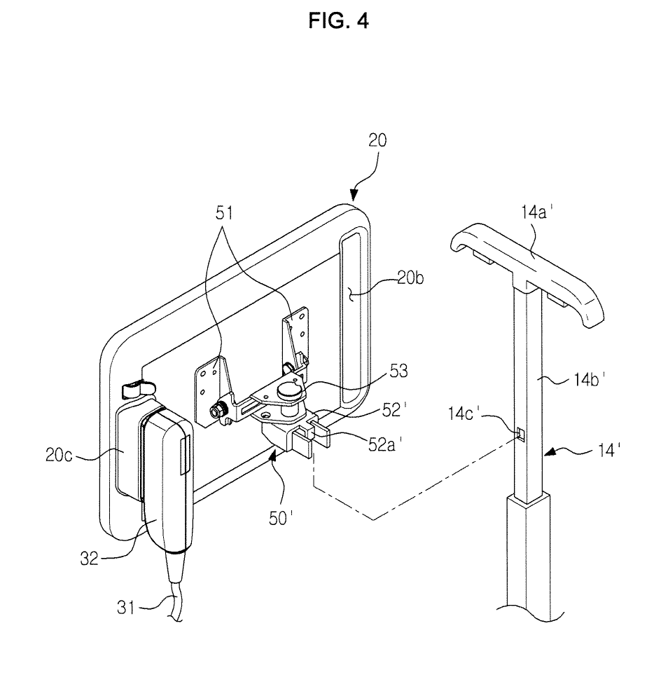

In addition, according to the present embodiment, the handle 14 includes a pair of elevation portions 14b. However, the disclosure is not limited thereto, and the handle 14 may include an elevation portion 14b′ and a handle portion 14a′ connected to the elevation portion 14b′ at the center as illustrated in FIG. 4.

According to the present embodiment, the control unit 12 is installed in the body 10. However, the disclosure is not limited thereto, and the control unit 12 may be integrally formed with the display unit 20.

According to the present embodiment, the probe 30 is connected to the display unit 20. However, the disclosure is not limited thereto, and the probe 30 may be connected to the body 10.

As is apparent from the above description, the portable ultrasonic diagnostic apparatus includes the display unit detachably mounted on the handle of the body. Since the handle is used as a stand supporting the display unit, the portable ultrasonic diagnostic apparatus may become more convenient to use.

Although a few embodiments of the present disclosure have been shown and described, it would be appreciated by those skilled in the art that changes may be made in these embodiments without departing from the principles and spirit of the invention, the scope of which is defined in the claims and their equivalents.

What is claimed is:

1. A portable ultrasonic diagnostic apparatus comprising:
a body;
a handle installed at a side of the body and configured for protruding from or retracting into the body; and
a display unit detachably mounted on the handle.

2. The portable ultrasonic diagnostic apparatus according to claim 1, further comprising wheels protruding downward from the body and supported by the ground.

3. The portable ultrasonic diagnostic apparatus according to claim 1, further comprising a probe connected to the body or the display unit, the probe transmitting ultrasonic waves to an object, and receiving ultrasonic waves from the object.

4. The portable ultrasonic diagnostic apparatus according to claim 1, wherein the body comprises a case defining an appearance of the body and the display unit is accommodated by the case.

5. The portable ultrasonic diagnostic apparatus according to claim 4, wherein the body comprises a control unit provided in the case.

6. The portable ultrasonic diagnostic apparatus according to claim 1, wherein:
the handle comprises a handle portion for gripping and at least one elevation portion that is vertically collapsable to the body and connected to the handle portion via an upper end; and
the display unit is detachably mounted on the elevation portion.

7. The portable ultrasonic diagnostic apparatus according to claim 6, wherein the at least one elevation portion comprises a pair of elevation portions extending in parallel to each other and connected to the handle portion via upper ends.

8. The portable ultrasonic diagnostic apparatus according to claim 7, further comprising a hinge assembly disposed between the display unit and the elevation portion to allow the display unit to be rotatably coupled to the handle.

9. The portable ultrasonic diagnostic apparatus according to claim 8, wherein the hinge assembly comprises a first hinge bracket mounted on the rear surface of the display unit, a second hinge bracket coupled to the handle, and a hinge member coupled to the first hinge bracket so as to be rotatable upward or downward and coupled to the second hinge bracket so as to be rotatable clockwise or counterclockwise.

10. The portable ultrasonic diagnostic apparatus according to claim 9, wherein:
the second hinge bracket comprises an engagement protrusion protruding backward and coupled to the elevation portion; and
the elevation portion has an engagement hole to which the engagement protrusion is coupled.

11. The portable ultrasonic diagnostic apparatus according to claim 4, wherein the case comprises a probe holder that rotatably protrudes from the case and holds the probe.

12. The portable ultrasonic diagnostic apparatus according to claim 11, wherein the probe holder has one end rotatably attached to a side of the case such that the probe holder is rotably collapsed to form a surface of the case, and protrudes rotatably from the case as the probe holder rotates.

13. The portable ultrasonic diagnostic apparatus according to claim 12, wherein the probe holder includes a plurality of probe holding grooves to hold the probe.

14. The portable ultrasonic diagnostic apparatus according to claim 1, wherein the display unit comprises a display disposed at the front surface of the display unit and displaying an image.

15. The portable ultrasonic diagnostic apparatus according to claim 14, wherein the display comprises a touch-screen.

16. The portable ultrasonic diagnostic apparatus according to claim 1, wherein the body further comprises an auxiliary handle disposed at one side of the body.

17. The portable ultrasonic diagnostic apparatus according to claim 4, wherein the case has a recessed platform on an upper surface of the case.
